(19) United States
(12) Patent Application Publication
(10) Pub. No.: US 2020/0390608 A1
(43) Pub. Date: Dec. 17, 2020

(54) EARPLUS ASSEMBLY AND METHOD FOR FORMING THE SAME

(71) Applicant: 3M INNOVATIVE PROPERTIES COMPANY, Saint. Paul, MN (US)

(72) Inventors: Jacob H. ELY, Carmel, IN (US); Kenneth F. TEETERS, Zionsville, IN (US); Robert C. COFFIN, Plainfield, IN (US); Feng CAI, Carmel, IN (US)

(73) Assignee: 3M Innovative Properties Company, Saint Paul, MN (US)

(21) Appl. No.: 16/643,120

(22) PCT Filed: Aug. 30, 2018
(86) PCT No.: PCT/IB2018/056647

§ 371 Date: Feb. 28, 2020

Related U.S. Application Data
(60) Provisional application No. 62/553,620, filed on Sep. 1, 2017.

(51) Int. Cl. A61F 11/12; A61L 31/04; A61L 31/14; B29C 65/20; B29C 65/56; B29C 65/74; B29C 65/00

(52) U.S. Cl. CPC A61F 11/12; A61F 2011/085; A61L 31/14; B29C 65/2061; B29C 65/565; B29C 65/7437; B29C 66/126; B29C 66/73116; B29C 66/73151; B29C 66/73921; B29C 66/12421; B29C 66/712; B29C 66/73152; B29C 66/81417; B29C 66/919; A61L 31/041

(57) ABSTRACT

An earplug assembly includes a first earplug, a second earplug, and a cord including a first end, a second end, each earplug including a stem and a sound attenuating body. A method for preparing the earplug assembly includes attaching the first and second ends of the cord to the first and second earplugs, respectively, by contacting a hot tip to the proximal end of the stem of the earplug to form an attachment point, removing the hot tip, and contacting or inserting the end of the cord to the attachment point to thermally bond the end of the cord to the stem of the earplug.

The present disclosure relates to hearing protection devices, e.g., push-to-fit earplugs, where two earplugs are connected by a cord, and methods of manufacturing the hearing protection devices.

The use of hearing protective and noise attenuating devices is well known, and various types of devices have been considered. Such devices include earplugs and semi-aural devices partially or completely constructed of foam or rubber materials that are inserted into, or placed over, the ear canal of a user to physically obstruct the passage of sound waves into the inner ear.

Compressible or “roll-down” type earplugs generally comprise a compressible, resilient body portion and may be made of suitable slow recovery foam materials. The earplug may be inserted into the ear canal of a user by first rolling it between fingers to compress the body portion, then pushing the body portion into the ear canal, and subsequently allowing the body portion to expand to fill the ear canal.

Push-to-fit type earplugs have also been considered, and may include a compressible attenuating portion and a stiff portion (e.g., a stem) that extends from the attenuating portion. To insert a push-to-fit type earplug, the user grasps the stiff portion and pushes the attenuating portion into the ear canal with an appropriate level of force. The attenuating portion compresses as it is accommodated in the ear canal. Push-to-fit earplugs may allow the earplug to be quickly and easily inserted in an ear canal, and may promote hygiene by minimizing contact with the attenuating portion of the earplug prior to insertion.

A pair of earplugs may be connected by a cord to form an earplug assembly. Although methods to attach the cord to the earplugs are known, they may be costly and may pose manufacturing challenges.

### SUMMARY

The present disclosure relates to earplug assemblies where two earplugs, e.g., push-to-fit earplugs, are connected by a cord, and methods of manufacturing earplug assemblies.

According to an embodiment, the earplug assembly comprises a first earplug, a second earplug, and a cord comprising a first end, a second end, and a length extending between the first and second ends, each earplug comprising a stem having a proximal end, a distal end, and a major outer surface and a longitudinal axis extending from the proximal end to the distal end; and a sound attenuating body disposed at the distal end of the stem. The method for preparing the earplug assembly comprises attaching the first and second ends of the cord to the first and second earplugs, respectively, by: inserting a hot tip to a depth into the proximal end of the stem of the earplug to form a cord cavity, the stem comprising a first material with a first melting temperature and the cord comprising a second material with a second melting temperature, and the hot tip having a temperature at or above the first and second melting temperatures; removing the hot tip from the stem; and inserting the end of the cord into the cord cavity.

According to another embodiment, the method comprises attaching the first and second ends of the cord to the first and second earplugs, respectively, by: contacting a hot tip with the proximal end of the stem of the earplug to melt a portion of the proximal end, the stem comprising a first material with a first melting temperature and the cord comprising a second material with a second melting temperature, and the hot tip having a temperature at or above the first and second melting temperatures; and contacting the end of the cord with the melted portion of the proximal end of the stem.

According one embodiment, an earplug assembly comprises a first earplug and a second earplug, each earplug comprising: a stem comprising a proximal end, a distal end, and a major outer surface and a longitudinal axis extending from the proximal end to the distal end; and a sound attenuating body disposed at the distal end of the stem. The earplug assembly further comprises a cord comprising a first end, a second end, and a length extending between the first and second ends, the first and second ends thermally bonded to and extending to a depth into the proximal ends of the stems of the first and second earplugs, the stem comprising a first material selected from polypropylene, polyethylene terephthalate, polybutylene terephthalate, nylon, polycarbonate, and mixtures thereof and the cord comprising a second material selected from thermoplastic polyolefins, thermoplastic elastomers, ethylene vinyl acetate, flexible polyvinyl chloride, styrene-butadiene copolymer, polypropylene-based elastomers, and mixtures thereof.

According to another embodiment, an earplug assembly comprises a first earplug and a second earplug, each earplug comprising: a stem comprising a proximal end, a distal end, and a major outer surface and a longitudinal axis extending from the proximal end to the distal end; and a sound attenuating body disposed at the distal end of the stem. The earplug assembly further comprises a cord comprising a first end, a second end, and a length extending between the first and second ends, the first and second ends thermally bonded to and extending to a depth into the proximal ends of the stems of the first and second earplugs by thermal bonding, the stem comprising a first thermoplastic having a first melting temperature and the cord comprising a second thermoplastic having a second melting temperature that is lower than the first melting temperature by at least 30° C.

The above summary is not intended to describe each embodiment or every implementation of the earplugs and methods of manufacturing earplugs as described herein. Rather, a more complete understanding of the invention will become apparent and appreciated by reference to the following Detailed Description and claims in view of the accompanying figures.

### BRIEF DESCRIPTION OF DRAWINGS

FIG. 1 shows an earplug assembly according to an embodiment.

FIG. 2A is a partial view of the earplug assembly of FIG. 1.

FIG. 2B is a partial cross-sectional view of the earplug assembly of FIG. 1.

FIGS. 3A-3D show a cartoon depiction of a method of making earplug assembly of FIG. 1 according to an embodiment.

### DETAILED DESCRIPTION

The present disclosure relates to earplugs, and more specifically to earplug assemblies including two earplugs connected by a cord. The present disclosure further relates to methods of making earplug assemblies with two earplugs and a cord. According to certain embodiments, the two earplugs of the earplug assembly are connected to the cord by thermal bonding. In some embodiments, the connection between the earplugs and the cord, or the earplug assembly as a whole, is free of adhesives.

The term “thermally bonded” means a state in which molecules of two materials or surfaces have diffused into the material or surface of the other when in a molten phase such that a bond is formed. Chemical bonding is absent or does not provide the primary source of bonding between thermally bonded materials or surfaces.

The term “thermoplastic” means a polymer that can be repeatedly heated and re-shaped and will retain its shape upon cooling.

The term “thermoset” is used to refer to polymers that are permanently and irreversibly cured and cannot remolded or reheated after their initial curing.

The term “melting temperature” is used to describe a temperature or temperature range at which a thermoplastic polymer transitions from a crystalline or semi-crystalline phase to an amorphous phase. Melting temperature can be determined using various test methods, such as differential scanning calorimetry (DSC).

As used here, the term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

As used here, all numbers are assumed to be modified by the term “about” and in certain situations, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

As used here, the recitations of numerical ranges by endpoints include all numbers subsumed within that range as well as the endpoints (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the invention. Thus, the appearances of such phrases in various places throughout this specification are not necessarily referring to the same embodiment of the invention. Furthermore, the particular features, configurations, compositions, or characteristics may be combined in any suitable manner in one or more embodiments.

Relative terms such as proximal, distal, left, right, forward, rearward, top, bottom, side, upper, lower, horizontal, vertical, and the like may be used in this disclosure to simplify the description, however, and not to limit the scope of the invention in any way. Terms such as left, right, forward, rearward, top, bottom, side, upper, lower, horizontal, vertical, and the like are from the perspective observed in the particular figure.

Earplugs that provide hearing protection for a user and methods of making earplug assemblies with two earplugs and a connecting cord are described herein. In the embodiments, various types of earplugs can be used as part of the earplug assembly. Although a certain type of earplug is shown in the Figures, this type should not be considered limiting, and other types and shapes of earplugs can be used.

In one or more embodiments, the earplug assembly includes two earplugs connected by a cord. One or more embodiments of methods of making earplug assemblies are also described. The cord may be attached to the earplugs by using a hot tip to create an attachment point (e.g., a cord cavity) at the proximal end of the stem and connecting the end of the cord with the attachment point (e.g., by inserting the end of the cord into the cord cavity) to thermally bond the cord to the earplug. In some embodiments, the attachment is free of adhesives.

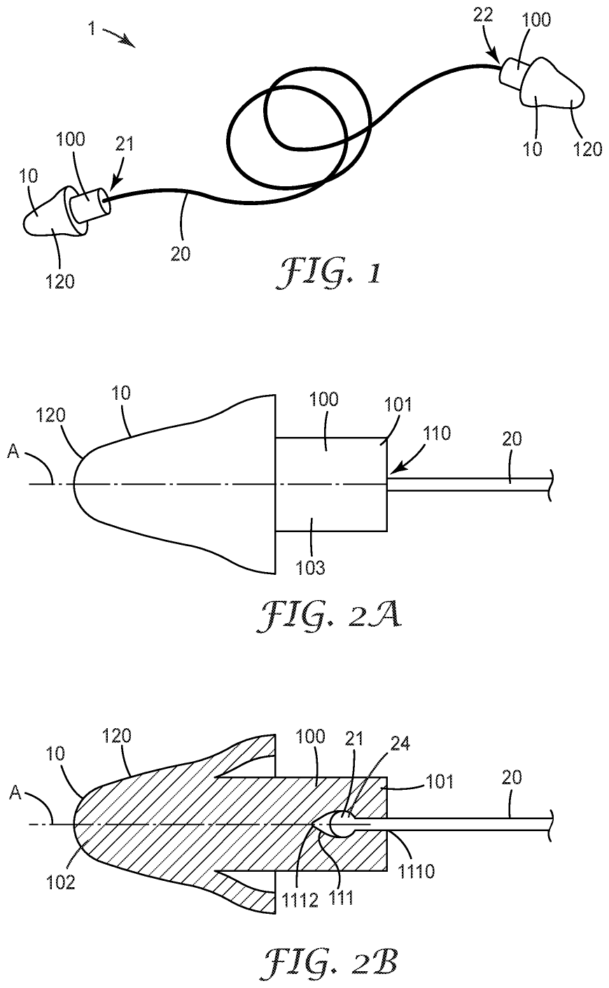

Referring now to FIG. 1, an earplug assembly 1 is shown. According to at least some embodiments, the earplug assembly 1 includes two earplugs 10 connected by a cord 20. While this disclosure discusses assemblies including two earplugs, an assembly with only a single earplug attached to a cord could be imagined and is considered to be within the scope of this disclosure.

Various types of earplugs can be used as part of the earplug assembly 1. For example, the earplugs may include a stem or central core, and may additionally include a sound-attenuating body. Alternatively, the earplug stem itself may function as the sound-attenuating body. The earplug assembly 1 shown in the FIGURES includes earplugs 10 with a stem 100 and a sound-attenuating body 120. The stem 100 has a proximal end 101, a distal end 102, and a major outer surface 103, and a longitudinal axis A extending from the proximal end 101 to the distal end 102. The proximal end 101 of the stem 100 is intended to be grasped by the user when inserting or removing the earplug 10 to and from the ear canal.

In the embodiment shown, a sound attenuating body 120 is attached to the major outer surface 103 at the distal end 102 of the stem 100. Generally, the sound-attenuating body 120 may be made of the same material as the stem 100, or a different material. The sound-attenuating body 120 may be made of a compressible material (e.g., a foam or an elastomer) that is suitable for inserting into the ear canal of the user.

The cord 20 has a first end 21 and a second end 22, and a length extending between the first and second ends 21, 22. The length of the cord is not limiting, and any useful cord length can be used. Typical cord lengths vary from about 12 to about 30 inches. According to an embodiment, the first and second ends 21, 22 are thermally bonded to the stems of the earplugs 10 of the assembly 1. In some embodiments, the attachment is free of adhesives, or if an adhesive is used, it does not provide the primary mechanism of adhesion between the cord and the earplugs.

FIGS. 2A and 2B show partial views of the earplug assembly, with FIG. 2B showing a cross-sectional view of the earplug 10 and one end of the cord 20. The cord 20 is attached to the earplug 10 at an attachment point 110 at the proximal end 101 of the stem 100. In certain embodiments, the attachment point 110 includes a cord cavity 111, and the end (e.g., first end 21 or second end 22) of the cord 20 extends into the cord cavity 111 and is thermally bonded to the material of the stem 100. A small air gap or cavity may remain at the bottom 1112 of the cord cavity 111 after the cord 20 is attached, as seen in FIG. 2B.

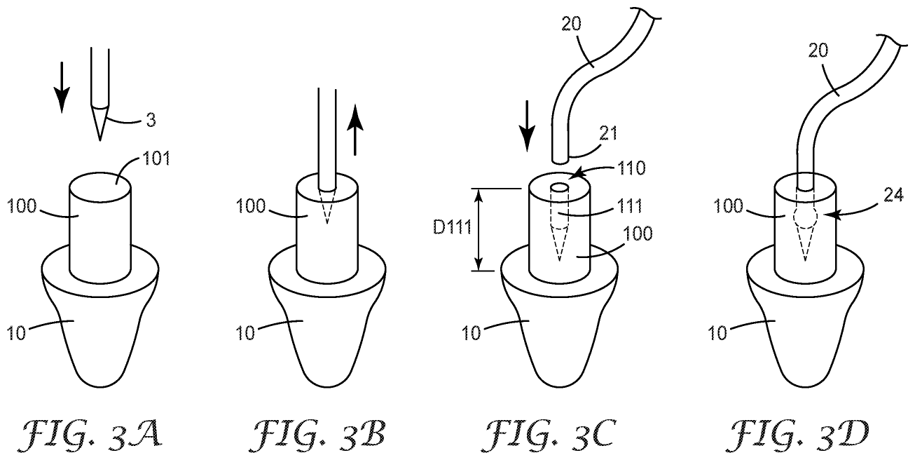

The primary mechanism of retaining the earplug at the end of the cord is thermal bonding. However, in some embodiments, the end of the cord 20 and the cord cavity 111 form a ball-and-socket attachment, where the end of the cord 20 forms a ball 24 that fits into the cavity 111 (the “socket”). The term “ball” is used here to indicate an enlarged end of the cord. Although a round ball is shown in FIGS. 2B and 3D, the ball 24 can have an irregular shape. The mouth 1110 of the cavity 111 may be narrower than the width of the ball 24, providing a friction fit in addition to the primary adhesion provided by thermal bonding.

The earplugs 10 and the cord 20 may be made of any suitable material. In some embodiments, the earplug stem 100 is made from a first material and the cord 20 is made from a second material. The second material may be different from the first material. For example, the first material may be selected to provide suitable characteristics for the stem, which may act as a handle for the earplug and as support for the sound-attenuating portion, or may act as the sound-attenuating portion. The second material may be selected to provide suitable characteristics for the cord, such as flexibility, comfort, durability, etc. Other aspects of the first and second materials may also be considered, such as compatibility with each other in thermal bonding, ease of processing, cost, and environmental considerations.

According to some embodiments, the stem 100 is made from a first material that may include suitable thermoplastic materials. For example, the first material may include polyolefins, such as polypropylene (PP) or polyethylene (PE), polyethylene terephthalate (PET), polybutylene terephthalate (PBT), poly(methyl methacrylate) (PMMA), acrylonitrile butadiene styrene (ABS), polyamide (nylon), polylactic acid (PLA), polycarbonate, polyether sulfone (PES), polyoxymethylene (POM), polyether ether ketone (PEEK), polyphenylene oxide (PPO), polyphenylene sulfide (PPS), polystyrene (PS), polyvinyl chloride (PVC), and mixtures and alloys thereof. In certain embodiments, the first material is polypropylene, polyethylene terephthalate, polybutylene terephthalate, nylon, polycarbonate, or a mixture or alloy thereof. The first material may be free of PVC.

The first material may be a rigid thermoplastic having a flexural modulus of about 0.3 to about 12 GPa, about 0.5 to about 10 GPa, or about 0.7 to about 8 GPa. In some embodiments, the first material forms a core that may be further surrounded by a foam material that also forms the sound-attenuating portion 120.

According to some embodiments, the cord 20 is made from a second material that may include suitable thermoplastic materials, such as thermoplastic polyolefins (TPOs) and thermoplastic elastomers (TPEs). For example, the second material may include ethylene vinyl acetate (EVA), flexible polyvinyl chloride (PVC), Styrene block copolymer (SBC), styrene-butadiene (SB) copolymer, polyurethane, polypropylene-based elastomers, and mixtures and alloys thereof.

The second material may be selected based on its hardness. For example, the second material may be selected from flexible thermoplastics that have type A shore hardness of about 25 to about 90, or about 30 to about 75, or about 35 to about 60. In certain embodiments the second material includes or is ethylene vinyl acetate (EVA). The second material may be free of PVC.

In some embodiments, the second material is selected to have a melting temperature (e.g., the second melting temperature) that is similar to (e.g., within about 10° C. of) or lower than the melting temperature of the first material (e.g., the first melting temperature). For example, the second material may have a melting temperature that is about 0 to about 200° C. lower, about 0 to about 150° C. lower, about 0 to about 100° C. lower, about 0 to about 75° C. lower, about 0 to about 50° C. lower, or about 0 to about 35° C. lower than the melting temperature of the first material. In one exemplary embodiment, the first material has a melting temperature of about 150 to about 200° C., and the second material has a melting temperature of about 70 to about 80° C. When melting temperatures of the two materials are compared, the same method (e.g., DSC) should be used to determine each melting temperature.

In embodiments described here, the cord is coupled with the stems of two earplugs to connect the earplugs in an earplug assembly that includes a first earplug, a second earplug, and a cord connecting the first and second earplugs, the cord having a first end, a second end, and a length extending between the first and second ends. One or more embodiments of methods of making earplug assemblies are also described. In one or more embodiments, the methods may reduce the difficulty and/or the cost of manufacturing earplug assemblies as described herein. For example, the methods may make manufacturing faster, simpler, and cheaper. The methods may, in one or more embodiments, include contacting an end of the earplug stem with a hot tip to create an attachment point for the cord, and attaching an end of the cord to the attachment point.

A simplified schematic of the method is shown in FIGS. 3A-3D. According to an embodiment, a hot tip 3 is contacted to the proximal end 101 of the stem 100 to melt a portion of the material of the stem 100 to create an attachment point 110. The hot tip 3 may contact the stem 100 approximately at the center of the proximal end 101. In some embodiments, the hot tip 3 is inserted into the stem 100 to a depth to create a cord cavity 111.

The hot tip 3 has a temperature that is above the first and second melting temperatures of the first and second materials. For example, the hot tip 3 may have a temperature of about 150 to about 450° C., about 200 to about 425° C., about 225 to about 400° C., or about 250 to about 350° C. The temperature of the hot tip 3 can be selected such that it is at least about 30° C., at least 40° C., at least 50° C., at least 75° C., or at least 100° C. above the melting temperature of the first material.

The hot tip 3 is removed from the stem 100 after a short period of time. The timing depends in part on the difference between the temperature of the hot tip 3 and the melting temperature of the first material. The greater the difference, the sooner the hot tip 3 can be removed. For example, when the difference is about 75° C. or greater, the hot tip 3 may be removed after about 0.1 to about 5 seconds, or about 0.2 to about 2 seconds. A greater length of time may be needed if the difference is smaller.

When the hot tip 3 contacts the proximal end 101 of the stem 100, it raises the temperature and melts a portion of the material of the stem 100. The end of the cord 20 can then be contacted with the melted portion at the attachment point 110. If the hot tip 3 was inserted into the stem 100 to create a cord cavity 111 with a depth D111, the end of the cord (e.g., first end 21 or second end 22) can be inserted into the cord cavity 111. The depth D111 of the cord cavity may be about 2 to about 10 mm, or about 3 to about 7 mm.

According to an embodiment, at the time when the cord 20 is contacted with the attachment point 110, the temperature of the melted portion of the stem 100 is higher than the melting temperature of the cord material (second material). The temperature of the melted portion may be sufficient to melt a portion of the end of the cord 20, thus thermally bonding the cord 20 to the earplug 10. In at least some embodiments, the heat source (e.g., the hot tip 3) is not applied to the cord, but only the stem of the earplug. Rather, the end of the cord 20 is melted by the residual heat of the stem 100. The end of the cord 20 can be contacted with the attachment point 110 soon after the hot tip 3 is removed and before the attachment point has cooled to a temperature below the melting temperature of the second material (the material of the cord 20).

The end of the cord 20 may be inserted all the way to the bottom of the cord cavity 111 (the insertion depth is equal to the depth D111 of the cord cavity 111). However, in some embodiments, the cord 20 is inserted only part of the way into the cord cavity 111 (the insertion depth is less than the depth D111 of the cord cavity 111). For example, the end of the cord 20 may inserted into the cord cavity 111 so that a small air gap or cavity remains at the bottom 1112 of the cord cavity 111 after the cord 20 is attached, as seen in FIG. 2B.

The end of the cord 20 can be held in place at the proximal end 101 of the stem 100 (e.g., in the cord cavity 111) for a length of time to allow for the thermal bonding to occur. For example, the cord 20 can be held in place for about 0.5 to about 4 seconds. After thermal bonding has occurred and the temperature of the material at the attachment point has cooled sufficiently (e.g., below the melting temperature of at least one of the materials), the cord will stay attached to the earplug without assistance (e.g., without holding).

In some embodiments, the melting of a portion of the end of the cord 20 may form a ball-and-socket type attachment of the cord in the cord cavity 111. As seen in FIGS. 2B and 3D, the end of the cord 20 may form an enlarged ball 24 inside the cord cavity 111, providing a friction fit in addition to the primary adhesion method of thermal bonding. The surrounding core material may at least partially conform to the shape of the ball 24.

### Exemplary Embodiments

Embodiment 1 is a method for preparing an earplug assembly, the earplug assembly comprising: a first earplug, a second earplug, and a cord comprising a first end, a second end, and a length extending between the first and second ends, each earplug comprising: a stem having a proximal end, a distal end, and a major outer surface and a longitudinal axis extending from the proximal end to the distal end; and a sound attenuating body disposed at the distal end of the stem, the method comprising attaching the first and second ends of the cord to the first and second earplugs, respectively, by: inserting a hot tip to a depth into the proximal end of the stem of the earplug to form a cord cavity, the stem comprising a first material with a first melting temperature and the cord comprising a second material with a second melting temperature, and the hot tip having a temperature at or above the first and second melting temperatures; removing the hot tip from the stem; and inserting the end of the cord into the cord cavity.

Embodiment 2 is the method of embodiment 1, wherein the inserting the end of the cord into the cord cavity melts a portion of the end of the cord.

Embodiment 3 is the method of embodiments 1 or 2, wherein the method forms a ball-and-socket attachment of the end of the cord in the cord cavity.

Embodiment 4 is the method of any of embodiments 1-3, further comprising holding the end of the cord in the cord cavity for about 0.5 to about 4 seconds.

Embodiment 5 is the method of any of embodiments 1-4, wherein the melting temperature of the second material is lower than the melting temperature of the first material by 20° C. or more, 30° C. or more, 50° C. or more, or 75° C. or more.

Embodiment 6 is the method of any of embodiments 1-5, wherein the first material is selected from rigid thermoplastics having a flexural modulus of about 0.3 to about 12 GPa, about 0.5 to about 10 GPa, or about 0.7 to about 8 GPa.

Embodiment 7 is the method of any of embodiments 1-6, wherein the first material comprises polypropylene, polyethylene terephthalate, polybutylene terephthalate, nylon, polycarbonate, or a mixture thereof.

Embodiment 8 is the method of any of embodiments 1-7, wherein the second material is selected from flexible thermoplastics having a type A shore hardness of about 25 to about 90, or about 30 to about 75, or about 35 to about 60.

Embodiment 9 is the method of any of embodiments 1-8, wherein the second material comprises thermoplastic polyolefins, thermoplastic elastomers, ethylene vinyl acetate, flexible polyvinyl chloride, styrene-butadiene copolymer, polypropylene-based elastomers, or a mixture thereof.

Embodiment 10 is the method of any of embodiments 1-9, wherein the hot tip has a temperature of about 175 to about 450° C., about 150 to about 450° C., about 200 to about 425° C., about 225 to about 400° C., or about 250 to about 350° C.

Embodiment 11 is the method of any of embodiments 1-10, wherein the hot tip is removed from the stem about 0.1 to about 5 seconds about 0.1 to about 2 seconds, or about 0.2 to about 2 seconds after inserting the hot tip.

Embodiment 12 is the method of any of embodiments 1-11, wherein the hot tip is inserted to a depth of about 2 to about 10 mm, or about 3 to 7 mm into the proximal end of the stem.

Embodiment 13 is a method for preparing an earplug assembly, the earplug assembly comprising: a first earplug, a second earplug, and a cord comprising a first end, a second end, and a length extending between the first and second ends, each earplug comprising: a stem having a proximal end, a distal end, and a major outer surface and a longitudinal axis extending from the proximal end to the distal end; and a sound attenuating body disposed at the distal end of the stem, the method comprising attaching the first and second ends of the cord to the first and second earplugs, respectively, by: contacting a hot tip with the proximal end of the stem of the earplug to melt a portion of the proximal end, the stem comprising a first material with a first melting temperature and the cord comprising a second material with a second melting temperature, and the hot tip having a temperature at or above the first and second melting temperatures; and contacting the end of the cord with the melted portion of the proximal end of the stem.

Embodiment 14 is the method of embodiment 13, further comprising holding the end of the cord to the melted portion of the stem for about 0.5 to about 4 seconds.

Embodiment 15 is the method of any of embodiments 13-14, wherein the melting temperature of the second material is lower than the melting temperature of the first material by 20° C. or more, 30° C. or more, 50° C. or more, or 75° C. or more.

Embodiment 16 is the method of any of embodiments 13-15, wherein the first material is selected from rigid thermoplastics having a flexural modulus of about 0.3 to about 12 GPa, about 0.5 to about 10 GPa, or about 0.7 to about 8 GPa.

Embodiment 17 is the method of any of embodiments 13-16, wherein the first material comprises polypropylene, polyethylene terephthalate, polybutylene terephthalate, nylon, polycarbonate, or a mixture thereof.

Embodiment 18 is the method of any of embodiments 13-17, wherein the second material is selected from flexible thermoplastics having a type A shore hardness of about 25 to about 90, or about 30 to about 75, or about 35 to about 60.

Embodiment 19 is the method of any of embodiments 13-18, wherein the second material comprises thermoplastic polyolefins, thermoplastic elastomers, ethylene vinyl acetate, flexible polyvinyl chloride, styrene-butadiene copolymer, polypropylene-based elastomers, or a mixture thereof.

Embodiment 20 is the method of any of embodiments 13-19, wherein the hot tip has a temperature of about 175 to about 450° C., about 150 to about 450° C., about 200 to about 425° C., about 225 to about 400° C., or about 250 to about 350° C.

Embodiment 21 is the method of any of embodiments 13-20, wherein the hot tip is removed from the stem 0.1 to about 5 seconds about 0.1 to about 2 seconds, or about 0.2 to about 2 seconds after inserting the hot tip.

Embodiment 22 is the method of any of embodiments 13-21, wherein the hot tip is inserted to a depth of about 2 to about 10 mm, or about 3 to 7 mm into the proximal end of the stem.

Embodiment 23 is an earplug assembly comprising: a first earplug and a second earplug, each earplug comprising: a stem comprising a proximal end, a distal end, and a major outer surface and a longitudinal axis extending from the proximal end to the distal end; and a sound attenuating body disposed at the distal end of the stem; and a cord comprising a first end, a second end, and a length extending between the first and second ends, the first and second ends thermally bonded to and extending to a depth into the proximal ends of the stems of the first and second earplugs, the stem comprising a first material selected from polypropylene, polyethylene terephthalate, polybutylene terephthalate, nylon, polycarbonate, and mixtures thereof and the cord comprising a second material selected from thermoplastic polyolefins, thermoplastic elastomers, ethylene vinyl acetate, flexible polyvinyl chloride, styrene-butadiene copolymer, polypropylene-based elastomers, and mixtures thereof.

Embodiment 24 is the earplug assembly of embodiment 23, wherein the attachment of the cord to the stem is free of adhesives.

Embodiment 25 is the earplug assembly of embodiments 23 or 24, wherein the first material comprises polypropylene, nylon, or a combination thereof.

Embodiment 26 is the earplug assembly of any of embodiments 23-25, wherein the second material is ethylene vinyl acetate (EVA).

Embodiment 27 is an earplug assembly comprising: a first earplug and a second earplug, each earplug comprising: a stem comprising a proximal end, a distal end, and a major outer surface and a longitudinal axis extending from the proximal end to the distal end; and a sound attenuating body disposed at the distal end of the stem; and a cord comprising a first end, a second end, and a length extending between the first and second ends, the first and second ends thermally bonded to and extending to a depth into the proximal ends of the stems of the first and second earplugs by thermal bonding, the stem comprising a first thermoplastic having a first melting temperature and the cord comprising a second thermoplastic having a second melting temperature that is lower than the first melting temperature by at least 20° C., 30° C. or more, 50° C. or more, or 75° C. or more.

Embodiment 28 is the earplug assembly of embodiment 27, wherein the attachment of the cord to the stem is free of adhesives.

Embodiment 29 is the earplug assembly of embodiments 27 or 28, wherein the first material comprises polypropylene, nylon, or a combination thereof.

Embodiment 30 is the earplug assembly of any of embodiments 27-29, wherein the second material is ethylene vinyl acetate (EVA).

### Example

A cord was attached to an earplug using the method described above. The earplug had a polypropylene (PP) composite core extending through the stem, surrounded by a layer of material that was foamed at the distal end to form a sound-attenuating portion. The polypropylene core was exposed at the proximal end of the stem. The cord was 1.27 mm thick ethylene vinyl acetate (EVA) with a melting temperature of approximately 74° C. The melting temperature of the PP core was in the range of 165-200° C.

A metal tip with a thickness of about 1.57 mm and a pointed end was heated to a temperature of about 370° C. and inserted into the proximal end of the PP core of the stem to create a cord cavity. The metal tip was held in the cord cavity for about 1 second, causing a portion of the core to melt. After the metal tip was removed from the cord cavity, the end of the cord was inserted into the cord cavity and was held in place for about 3 seconds.

It was observed that the end of the cord was thermally bonded to the stem of the earplug. It was further observed that a portion of the cord had melted and formed a ball inside the cord cavity, forming a ball-and-socket type connection. The connection between the cord and the earplug was found to be sufficiently strong for use in earplug assemblies without the use of adhesives.

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A method for preparing an earplug assembly, the earplug assembly comprising:
a first earplug, a second earplug, and a cord comprising a first end, a second end, and a length extending between the first and second ends, each earplug comprising:
a stem having a proximal end, a distal end, and a longitudinal axis extending from the proximal end to the distal end; and
a sound attenuating body disposed at the distal end of the stem,
the method comprising attaching the first and second ends of the cord to the first and second earplugs, respectively, by:
inserting a hot tip to a depth into the proximal end of the stem of the earplug to form a cord cavity, the stem comprising a first material with a first melting temperature and the cord comprising a second material with a second melting temperature, and the hot tip having a temperature at or above the first and second melting temperatures;
removing the hot tip from the stem; and
inserting the first or second end of the cord into the cord cavity.

2. The method of claim 1, wherein the inserting the first or second end of the cord into the cord cavity melts a portion of the end of the cord.

3. The method of claim 1, wherein the method forms a ball-and-socket attachment of the end of the cord in the cord cavity.

4. The method of claim 1, further comprising holding the end of the cord in the cord cavity for about 0.5 to about 4 seconds.

5. The method of claim 1, wherein the melting temperature of the second material is lower than the melting temperature of the first material by 50° C. or more.

6. The method of claim 1, wherein the first material is selected from rigid thermoplastics having a flexural modulus of about 0.3 to about 12 GPa.

7. The method of claim 1, wherein the first material comprises polypropylene, polyethylene terephthalate, polybutylene terephthalate, nylon, polycarbonate, or a mixture thereof.

8. The method of claim 1, wherein the second material is selected from flexible thermoplastics having a type A shore hardness of about 25 to about 90.

9. The method of claim 1, wherein the second material comprises thermoplastic polyolefins, thermoplastic elastomers, ethylene vinyl acetate, flexible polyvinyl chloride, styrene-butadiene copolymer, polypropylene-based elastomers, or a mixture thereof.

10. The method of claim 1, wherein the hot tip has a temperature of about 175 to about 425° C.

11. The method of claim 1, wherein the hot tip is removed from the stem about 0.1 to about 2 seconds after inserting the hot tip.

12. The method of claim 1, wherein the hot tip is inserted to a depth of about 2 to about 10 mm into the proximal end of the stem.

13. (canceled)

14. An earplug assembly comprising:
a first earplug and a second earplug, each earplug comprising:
a stem comprising a proximal end, a distal end, and a longitudinal axis extending from the proximal end to the distal end; and
a sound attenuating body disposed at the distal end of the stem; and
a cord comprising a first end, a second end, and a length extending between the first and second ends, the first and second ends thermally bonded to and extending to a depth into the proximal ends of the stems of the first and second earplugs, the stem comprising a first material selected from polypropylene, polyethylene terephthalate, polybutylene terephthalate, nylon, polycarbonate, and mixtures thereof and the cord comprising a second material selected from thermoplastic polyolefins, thermoplastic elastomers, ethylene vinyl acetate, flexible polyvinyl chloride, styrene-butadiene copolymer, polypropylene-based elastomers, and mixtures thereof.

15. The earplug assembly of claim 14, wherein the attachment of the cord to the stem is free of adhesives.

16. The earplug assembly of claim 14, wherein the first material comprises polypropylene, nylon, or a combination thereof.

17. The earplug assembly of claim 14, wherein the second material is ethylene vinyl acetate (EVA).

18. An earplug assembly comprising:
a first earplug and a second earplug, each earplug comprising:
a stem comprising a proximal end, a distal end, and a longitudinal axis extending from the proximal end to the distal end; and
a sound attenuating body disposed at the distal end of the stem; and
a cord comprising a first end, a second end, and a length extending between the first and second ends, the first and second ends thermally bonded to and extending to a depth into the proximal ends of the stems of the first and second earplugs by thermal bonding, the stem comprising a first thermoplastic having a first melting temperature and the cord comprising a second thermoplastic having a second melting temperature that is lower than the first melting temperature by at least 30° C.

19. The earplug assembly of claim 18, wherein the attachment of the cord to the stem is free of adhesives.

20. The earplug assembly of claim 18, wherein the first material comprises polypropylene, nylon, or a combination thereof.

21. The earplug assembly of claim 18, wherein the second material is ethylene vinyl acetate (EVA).
